(19) United States
(12) Patent Application Publication
(10) Pub. No.: US 2007/0179477 A1
(43) Pub. Date: Aug. 2, 2007

(54) Surgical coupling device

(76) Inventor: Karl-Heinz Danger, Detmold (DE)

Correspondence Address: Bliss McGlynn, P.C., Suite 600, 2075 West Big Beaver Road, Troy, MI, 48084, US

(21) Appl. No.: 11/652,428

(22) Filed: Jan. 11, 2007

(30) Foreign Application Priority Data
Jan. 12, 2006 (DE) 10 2006 001 677.7

(51) Int. Cl. A61B 17/00

(52) U.S. Cl. 606/1

(57) ABSTRACT

A surgical coupling device detachably connects a tool holder to a surgical instrument, wherein the tool holder is provided with a recess into which a coupling portion of the instrument can be inserted detachably, wherein a locking recess is provided in an inner wall of the recess, with which a locking member can be engaged detachably, the locking member being supported at the instrument, wherein the locking member is selectively movable into a locking position and a release position by a servo drive.

### BACKGROUND OF THE INVENTION

1. Field of the Invention

The present invention relates generally to surgical devices and, more specifically, to a surgical coupling device for detachably connecting a tool holder to a surgical instrument.

2. Description of the Related Art

Surgical instruments, which are inserted into a tool holder, are known in various embodiments. The tool holders are usually connected to a drive such that tools or parts of the surgical instruments can be driven, e.g. can be displaced in rotation. Such surgical instruments may, for example, be dental drills or cutters or they may also be formed as shaver instruments for arthroscopy.

From the state of the art, it is known (see e.g. EP 1 006 898 B1) to provide the tool holder with a centric bore or recess into which a coupling portion or shoulder portion of the surgical instrument is inserted. Therewith, corresponding driving couplings of the tool holder engage with coupling parts of the surgical instrument, such that a rotary drive is realized. The insertion and locking of the surgical instrument in the tool holder is achieved in that the tool holder is provided with a locking recess with which a locking member of the surgical instrument can be engaged detachably. The locking recess may, for example, be formed as an annular groove or be provided as only singular. The locking member of the surgical instrument may, for example, be formed as a snap-in hook, a notch, or the like. When inserting the surgical instrument into the tool holder, a locking or coupling may thus be obtained, which prevents an unintended detaching of the surgical instrument from the tool holder. In order to remove the surgical instrument from the tool holder, for example, an actuator is operated.

The devices known from the state of the art are often structured intricately and their manufacturing is correspondingly cost-intensive and elaborate. These device are often poorly operable under surgical operating conditions.

### SUMMARY OF THE INVENTION

It is an object of the present invention to provide a surgical coupling device of the aforementioned kind, which can be operated safely and easily while being simply structured and manufactured simply and cost-effectively.

According to the present invention, the object is solved by the combination of features of the present invention.

According to the present invention, the locking member can be operated by a servo drive. The servo drive may, according to the present invention, be for example formed as a servomotor or a solenoid. Therewith, there results the decisive advantage that the user of the tool holder does not have to apply any additional locking or releasing force when inserting or removing the instrument. This is particularly advantageous for a surgical use, as in this case the sensitive handling of levers, knobs, rasters or the like is often not possible or only difficult to perform due to the use of surgical gloves.

According to the present invention, there results a simple handling which additionally increases the operating reliability considerably.

In an advantageous further development of the present invention, the servo drive can be operated manually. However, in another embodiment, it may also be operated by a sensor, wherein the sensor enables an automatic or semi-automatic actuation of the servo drive. It is, for example, possible to bring the locking member automatically into its locked position when the instrument, upon inserting the instrument into the tool holder, is inserted correctly. For releasing the instrument, it is then only necessary to actuate the servo drive, be it manually or automatically. Such an automatic actuation of the servo drive may also be coupled with a control program for the tool holder or the instrument, respectively. The same can switch-off the drive when the instrument is to be replaced, wherein, in this case, the locking member is correspondingly brought automatically into its release position, such that the operator can replace the instrument.

According to the present invention, there also results the possibility to handle the instrument by a surgery robot and to replace the same automatically.

It should be appreciated that an automatic operation and a manual operation of the servo drive can be provided simultaneously. In case of a manual operation, for example, a pushbutton or a switch can be provided, which opens or closes the locking member by simply pushing the same (moves the same into its locked position or into its release position).

According to the present invention, the servo drive may comprise a servomotor which may be accommodated in the instrument or in the tool holder. It should be appreciated that the present invention may also comprise a mechanical reversal. According to the present invention, also mixed constructions are possible, in which the locking member is disposed at the instrument, while the instrument is actuated by a servo drive which is located at the tool holder.

In place of a servomotor, a solenoid may also be provided, which switches the locking member between the two positions (locked position and release position), as mentioned above. In this case, it is possible to form the locking member as a part of the solenoid, such that, for example, the core of the solenoid takes over the function of the locking member. Herewith, a cost-effective and simple embodiment can be realized.

The energy supply of the servo drive can be a battery which is attached to the instrument. It is also possible to provide energy through the tool holder, either directly or by interconnecting a secondary battery in the instrument. Due to the inventive embodiment, it is further possible to maintain either the locked position or the release position of the locking member in an electroless state of the servo drive. Therewith, the inventive embodiment may provide an additional degree of operating safety, in case that a failure of the energy supply should occur during operation.

In a further development of the present invention, it is possible to provide a plurality of instruments in an instrument dispenser. Therewith, the operator may single-handedly remove the individual instruments from the instrument dispenser and couple the same with the tool holder or deposit the instruments again into the instrument dispenser. Herefrom, there results a high degree of operating safety and ease of use. Due to the possibility to control the servo drive by a sensor, the instrument may be released as soon as it is inserted completely into the instrument dispenser. Analogously, a new instrument may be locked automatically after having been received completely, before it is removed from the instrument dispenser.

Due to the described embodiment, it is also possible to determine the correct and predetermined sequence of individual instruments for an operation. Therewith, the surgeon is prevented from inserting the wrong instrument into the tool holder. Furthermore, an additional quality check of the individual instruments may be performed, such that, for example, a re-insertion of an already used, blunt instrument can be prevented. Therewith, for example, thermal damages of the patient's tissue can be avoided.

It should be appreciated that the manual operation of the servo drive comprises different types of pushbuttons, switches, etc., such that also foot switches or the like are possible, which may be coupled with additional actuators, for example, for driving the instrument.

Other objects, features, and advantages of the present invention will be readily appreciated, as the same becomes better understood, after reading the subsequent description taken in conjunction with the accompanying drawings.

### BRIEF DESCRIPTION OF THE DRAWINGS

In the following, the present invention is described on the basis of an example in combination with the drawing, in which:

FIG. 1 shows a schematic partial section of the inventive coupling device in a locked state, and

FIG. 2 shows a view, analogous to FIG. 1, in a released state.

### DESCRIPTION OF THE PREFERRED EMBODIMENT(S)

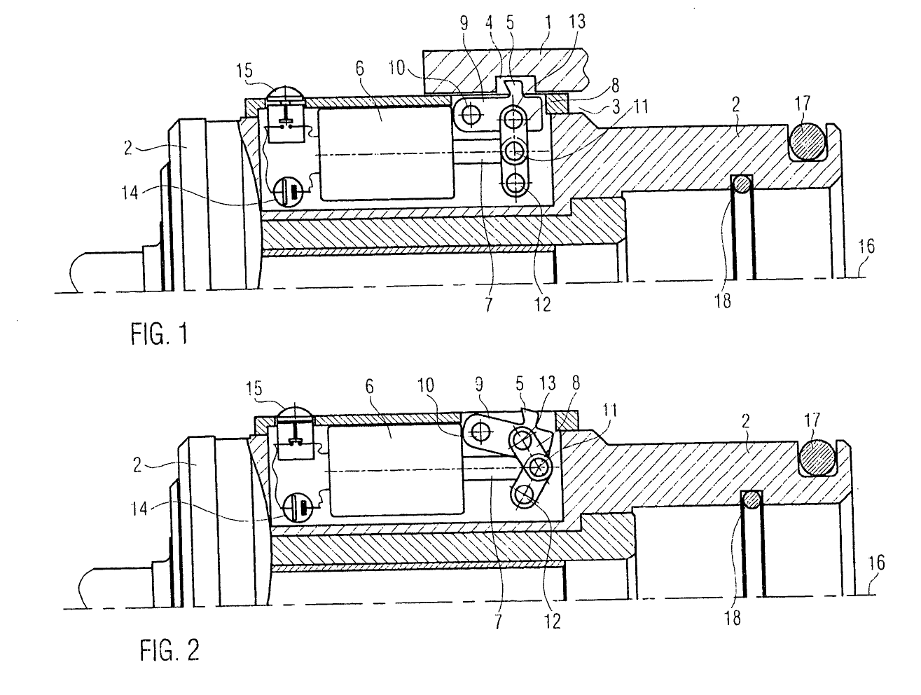

Referring now to the drawings, and in particular FIG. 1, one embodiment of a surgical coupling device, according to the present invention, is shown. FIG. 1 shows, strongly simplified, a part of a surgical tool holder 1 into which a surgical instrument 2 can be inserted. The insertion of the instrument 2 takes place in a recess 3 of the tool holder 1. The tool holder 1 is provided with a locking recess 4 which may be formed as an annular groove or as a singular recess in an inner wall of the recess 3.

A hook-shaped locking member 5 may be inserted into the locking recess 4. In the embodiment illustrated, the position of the locking member 5 may be selected by a servo drive 6. The servo drive 6 may be formed as an electromotor, servomotor, or a solenoid. The actuation of the locking member 5 is performed through a rod 7 which is coupled to a locking lever 9 through a knee-lever mechanism 8. The locking lever 9 is integrally provided with the locking member 5 and pivotable around a stationary rotary axis 10. At the free end of the rod 7, a rotary axis 11 is provided, with which the two levers of the knee-lever mechanism 8 are pivotally coupled to the rod 7. The lower lever of the knee-level mechanism 8 is supported pivotally at a stationary rotary axis 12, whereas the upper lever is pin-jointed with the locking lever 9 at a rotary axis 13.

The servo drive 6 is provided with a battery 14. Furthermore, a pushbutton 15 is provided, which is manually operable to operate the servo drive 6. It should be appreciated that it is also possible to provide energy through the tool holder 1, either directly or by interconnecting a secondary battery in the surgical instrument 2. It should also be appreciated that the manual operation of the servo drive 6 may comprise different types of pushbuttons, switches, etc., such that also foot switches or the like are possible, which may be coupled with additional actuators, for example, for driving the surgical instrument 2.

The figures furthermore show washers 17 and 18 as well as the center axis 16 of the coupling device.

FIG. 1 shows the locking member 5 in the locked position. In this position, the knee-lever mechanism 8 is stilted to the left by the movement of the rod 7. Therewith, there results a secure support of the locking member 5. In order to release the locking member 5 and to move the same into the release position shown in FIG. 2, the rod 7 is displaced to the right by the servo drive 6. Consequently, the knee-lever mechanism 8 dislocates the locking lever 9, as it is shown in FIG. 2. It should be appreciated that the locking member 5 may be maintained in the locked position in an electroless state of the servo drive 6. It should also be appreciated that the locking member 5 may be maintained in the release position in an electroless state of the servo drive 6.

In another embodiment of the present invention, the surgical coupling device may be operated by a sensor in place of the switch 15, wherein the sensor enables an automatic or semi-automatic actuation of the servo drive 6. The sensor may be arranged at the tool holder 1, the surgical instrument 2, or separately from either the tool holder 1 or surgical instrument 2.

In operation, the locking member 5 is brought automatically into its locked position when the surgical instrument 2, upon inserting the surgical instrument 2 into the tool holder 1, is inserted correctly as sensed by the sensor. For releasing the surgical instrument 2, it is then only necessary to actuate the servo drive 6 automatically. Such an automatic actuation of the servo drive 6 may also be coupled with a control program for the tool holder 1 or the surgical instrument 2, respectively. The sensor can switch-off the servo drive 6 when the surgical instrument 2 is to be replaced, wherein, in this case, the locking member 5 is correspondingly brought automatically into its release position, such that the operator can replace the surgical instrument 2.

The present invention has been described in an illustrative manner. It is to be understood that the terminology, which has been used, is intended to be in the nature of words of description rather than of limitation.

Many modifications and variations of the present invention are possible in light of the above teachings. Therefore, within the scope of the appended claims, the present invention may be practiced other than as specifically described.

What is claimed is:

1. A surgical coupling device for detachably connecting a tool holder to a surgical instrument, wherein the tool holder is provided with a recess into which a coupling portion of the instrument can be inserted detachably, wherein
a locking recess is provided in an inner wall of the recess, said surgical coupling device comprising:
a locking member engaged detachably with the locking recess, said locking member being supported by the surgical instrument; and
a servo drive to selectively move said locking member into a locking position and a release position with the locking recess.

2. A surgical coupling device as set forth in claim 1 wherein said servo drive comprises a servomotor.

3. A surgical coupling device as set forth in claim 1 wherein said servo drive comprises a solenoid.

4. A surgical coupling device as set forth in claim 1 wherein said servo drive is operated manually.

5. A surgical coupling device as set forth in claim 1 including a sensor to operate said servo drive.

6. A surgical coupling device as set forth in claim 5 wherein said sensor is arranged at the tool holder.

7. A surgical coupling device as set forth in claim 5 wherein said sensor is arranged at the surgical instrument.

8. A surgical coupling device as set forth in claim 5 wherein said sensor is arranged separately from the tool holder and the surgical instrument.

9. A surgical coupling device as set forth in claim 1 wherein said servo drive is operated automatically coupled with a control of the tool holder.

10. A surgical coupling device as set forth in claim 1 wherein said locking member is maintained in the locking position in an electroless state of said servo drive.

11. A surgical coupling device as set forth in claim 1 wherein said locking member is maintained in the release position in an electroless state of said servo drive.

12. A surgical instrument assembly comprising:
a surgical instrument having a coupling portion;
a tool holder having a recess to receive said coupling portion of said surgical instrument, said coupling portion being inserted detachably into said recess;
said recess having an inner wall and a locking recess in said inner wall;
a locking member engaged detachably with said locking recess, said locking member being supported at said surgical instrument; and
a servo drive to selectively move said locking member into a locking position and a release position with said locking recess.
